(19) United States
(12) Patent Application Publication
(10) Pub. No.: US 2010/0069710 A1
(43) Pub. Date: Mar. 18, 2010

(54) TREATMENT METHOD

(76) Inventors: Ken Yamatani, Tokyo (JP); Takayasu Mikkaichi, Tokyo (JP); Kazushi Murakami, Tokyo (JP); Kosuke Motai, Tokyo (JP); Ayano Ishioka, Tokyo (JP); Kunihide Kaji, Tokyo (JP)

Correspondence Address: OSTROLENK FABER GERB & SOFFEN, 1180 AVENUE OF THE AMERICAS, NEW YORK, NY, 100368403, US

(21) Appl. No.: 12/552,405

(22) Filed: Sep. 2, 2009

Related U.S. Application Data
(60) Provisional application No. 61/093,490, filed on Sep. 2, 2008.

(51) Int. Cl. A61B 1/00; A61B 1/018

(52) U.S. Cl. 600/102; 600/106

(57) ABSTRACT

A treatment method for a tissue in a body cavity of a patient includes the steps of endoscopically introducing treatment tools into the body cavity of the patient from a natural opening of the patient, and setting the posture of the patient to a prone position.

### BACKGROUND OF THE INVENTION

1. Field of the Invention

The present invention relates to a treatment method in which a medical device is inserted into a body cavity and treatment is performed on various tissues in the body cavity.

2. Description of the Related Art

Conventionally, various manipulations, such as extirpation of a gallbladder, are performed using a laparoscope or the like, as an example of less-invasive treatment. Such a laparoscopy is performed by making a plurality of holes in an abdominal wall, and inserting a plurality of instruments into the holes.

In recent years, in order to reduce the number of the holes made in the abdominal wall to reduce a patient's burden, a method of inserting a soft endoscope from natural openings, such as the mouth, nose or anus of a patient, to perform manipulation is suggested. As medical devices used for such manipulation, for example, a treatment endoscope as described in United States Patent Application, Publication No. 2007-0249897 is suggested. This treatment endoscope has a soft insertion portion which has flexibility, and the tip of the insertion portion is provided with a pair of arm portions having bent portions which perform a bending operation so that a plurality of channels arranged in the insertion portion are communicated with inner cavities of the arm portions, respectively. An operating portion of the treatment endoscope is connected to the arm portions by an operating member so that the arm portions can be operated to be bent vertically and horizontally.

An operator can perform manipulation on a tissue to be treated, substantially similarly to a laparoscopy, by inserting a treatment tool, such as forceps, into the channels, mounting an operating portion of the treatment tool on the operating portion of the treatment endoscope, making the tip of the treatment tool project from the arm portion, and operating the operating portion vertically and horizontally.

### SUMMARY OF THE INVENTION

A treatment method according to a first aspect of the present invention is a treatment method for a tissue in a body cavity of a patient including the steps of endoscopically introducing a treatment tool into the body cavity of the patient from a natural opening of the patient, and setting the posture of the patient to a prone position.

A treatment method according to a second aspect of the present invention is a treatment method for a tissue in a body cavity of a patient. The treatment method includes the steps of changing the posture of the patient to move organs in a thoracic cavity and an abdominal cavity of the patient so as to form a space, endoscopically introducing a treatment tool into the body cavity of the patient from a natural opening of the patient or a pierced hole formed in the body wall of the patient, and making the treatment tool approach organs in at least one of the thoracic cavity and the abdominal cavity through the space.

An operation method according to one aspect of the present invention includes the step of introducing a plurality of endoscopes into a body cavity of a patient.

### BRIEF DESCRIPTION OF THE DRAWINGS

FIG. 1 is a flow chart showing the flow of a treatment method of the present invention.

FIG. 2 is a drawing showing an insertion portion of a treatment endoscope which can be used for the treatment method.

FIG. 3 is a view showing the operation when the treatment endoscope is used.

FIG. 4 is a view showing an example of a postural change in a treatment method according to a first embodiment of the present invention.

FIG. 5 is a view showing an example of a postural change in a treatment method according to a second embodiment of the present invention.

FIG. 6 is a view showing an example of a postural change in a treatment method according to a third embodiment of the present invention.

FIGS. 7A to 7D are views showing the flow of a manipulation of a tunneling method.

FIG. 8 is a view showing an example of a postural change in a treatment method according to a fourth embodiment of the present invention.

FIG. 9 is a view showing an example of a postural change in another example of the treatment method.

FIGS. 10A to 10D are views showing the flow of manipulation when the treatment method is applied to a Nissen operation.

FIG. 11 is a view showing an example of a postural change in a treatment method according to a fifth embodiment of the present invention.

FIG. 12 is a view showing an example of a first postural change in a treatment method according to a sixth embodiment of the present invention.

FIG. 13 is a view showing an example of a second postural change in the treatment method.

FIG. 14 is a view showing an example of a first postural change in a treatment method according to a seventh embodiment of the present invention.

FIG. 15 is a view showing an example of a second postural change in the treatment method.

FIG. 16 is a view showing another example of the treatment method of the present invention.

### PREFERRED EMBODIMENTS

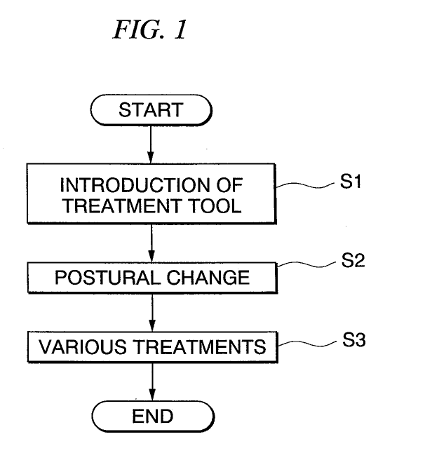

Hereinafter, a treatment method of a first embodiment of the present invention will be described with reference to FIGS. 1 to 4.

FIG. 1 is a flow chart showing the flow of a treatment method according to the present invention. The treatment method of this embodiment includes Step S1 of introducing a treatment tool into a body cavity of a patient or the like, Step S2 of setting the posture of a patient to a predetermined state to move an organ within the body cavity, and Step S3 of performing various treatments on a tissue within the body cavity using the treatment tool inserted into the body cavity.

As the treatment tool inserted into the body cavity in Step S1, various kind of treatment tools, such as grip forceps and a high-frequency knife, may be suitably selected and used according to a treatment to be performed.

A treatment endoscope described in the United States Patent Application, Publication No. 2007/0249897 can be suitably used for making these treatment tools reach a tissue to be treated. Although the details of the structure of the endoscope are described in the United States Patent Application, Publication No. 2007/0249897, the outline of the structure will be described below.

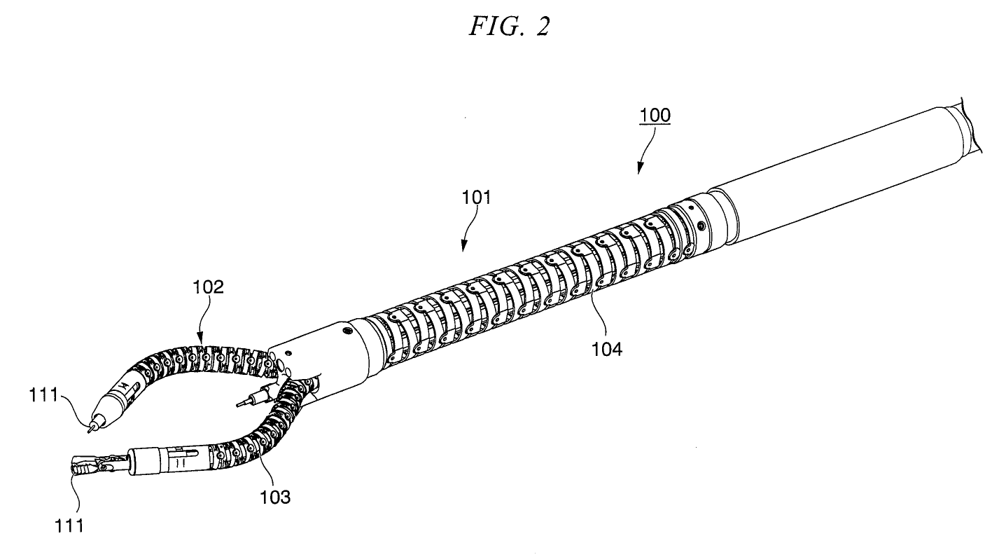

FIG. 2 is a perspective view showing an insertion portion 101 of the above-described treatment endoscope 100 which is inserted into a body cavity. The insertion portion 101 has flexibility, and has a pair of arm portions 102 and an observation device (not shown) for observing a tissue in the body cavity, the pair of arm portions 102 and the observation device being attached to the tip of the insertion portion. Additionally, the insertion portion 101 is provided with two operating channels (not shown) for inserting various kinds of treatment tools, such as grip forceps and a high-frequency knife. The pair of arm portions 102 are in the shape of a tube which has an inner cavity, and the inner cavities communicate with the operating channels, respectively. Accordingly, it is possible to insert various treatment tools into the operating channels of the insertion portion 101, and make the tips thereof project from the tips of the arm portions 102, as shown in FIG. 2.

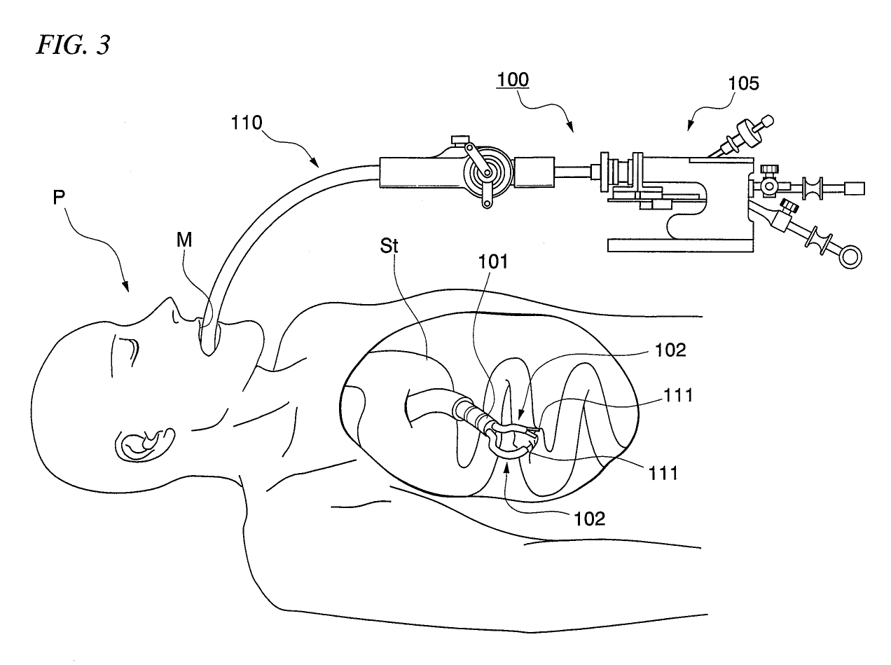

The arm portion 102 is configured such that a plurality of joint rings 103 are connected together so as to be aligned in an axial direction thereof. The insertion portion 101 is also configured such that a plurality of joint rings 104 are connected together so as to be aligned in an axial direction thereof. One end of an operating member, such as a wire, is connected to the joint rings 103, and the other end of the operating member is connected to an arm operating portion 105 shown in FIG. 3. Similarly, an operating member is connected to the joint rings 104 and an endoscope operating portion (not shown).

By the above-described configuration, the arm portions 102 and the insertion portion 101 can be operated to be bent vertically and horizontally with respect to their own axes by operating the arm operating portion 105 and the endoscope operating portion, respectively.

Accordingly, as shown in FIG. 3, the treatment endoscope 100 is inserted into a body cavity of a patient P or the like from a natural opening, such as a mouth M, and incises the wall surface of a luminal tissue, such as a stomach St, so that the endoscope can approach various tissues within an abdominal cavity or a thoracic cavity. In addition, in FIG. 3, although the treatment endoscope is inserted through an over-tube 110 and inserted into a body cavity, the over-tube 110 is not indispensable to the treatment method of this embodiment, and may be appropriately used as necessary.

Next, Step S2 of setting the posture of a patient to a predetermined state to move an organ within a body cavity will be described using an example in which the excision of a pancreas is performed by using the above-described treatment endoscope 100.

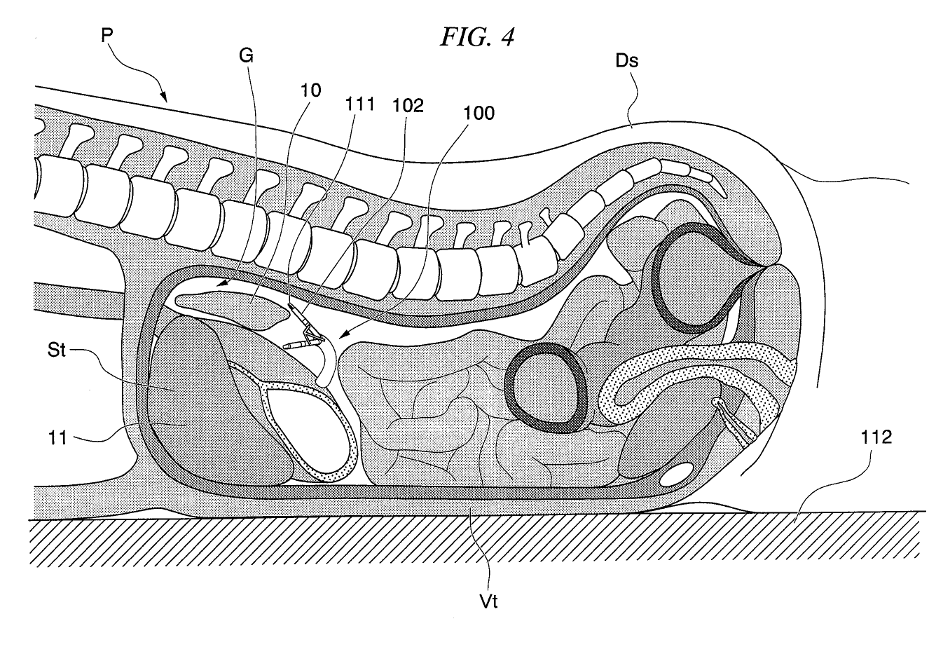

In Step S1, an operator introduces the treatment tools 111 into the abdominal cavity from the stomach St as shown in FIG. 3, using the treatment endoscope 100, and starts manipulation, such as membrane peeling, on a part of a pancreas 10 (refer to FIG. 4) to be excised. At this time, the patient P still maintains a supine position which is a position at the time of anesthesia induction.

Since the pancreas 10 is on the back side, the pancreas is brought into close contact with the stomach St, the gap between the stomach St and the pancreas 10 is narrow, and manipulation becomes increasingly difficult. Thus, in Step S2, as shown in FIG. 4, the operator converts the posture of the patient P on a bed 112, and sets the posture to a prone position, thereby turning the back side Ds up.

By turning the back side Ds up, the stomach St or a liver 11 moves to a lower belly side Vt due to gravity. On the other hand, since a number of regions of the pancreas 10 are fixed to the back side, the gap (space) G is formed between the pancreas 10 and the stomach St or the like, which results in an environment where manipulation on the pancreas 10 is easily performed.

In Step S3, the operator advances the tips of the arm portions 102 of the treatment endoscope 100 to the gap G, and performs manipulation (treatment) on the pancreas 10 by the treatment tools 111 which have projected from the tips of the arm portions 102, thereby excising a desired region (or whole pancreas 10). The excised tissue is collected by pulling out the treatment endoscope 100 to the outside of the body, while being gripped by the treatment tools 111.

According to the treatment method of this embodiment, in Step S2, since a gap is secured between the pancreas 10 to be treated, and other organs or tissues, such as the stomach St, by converting and setting the posture of the patient P from a supine position to a prone position, manipulation can be more easily performed.

In a conventional treatment method of opening a hole in an abdominal wall to insert treatment tools or a laparoscope, since the hole of the abdominal wall cannot be used when the patient takes a prone position, it is necessary to further open a hole in the back side. It is also necessary to close the hole of the abdominal wall before the patient takes the prone position, and such posture setting is practically impossible.

In the treatment method of this embodiment, the treatment tools 111 used for treatment are introduced via a natural opening, such as the mouth M, using the treatment endoscope 100 which has flexibility. Therefore, since it is not necessary to form a new hole or to close the hole once formed at the time of a postural change, the time required for treatment can be shortened, and any invasion imposed on the patient can be reduced.

Additionally, the treatment endoscope 100 and the treatment tools 111 which have flexibility have little force for acting on a tissue at the time of manipulation as compared with a so-called hard treatment tool in which an insertion portion does not have flexibility, therefore it is not easy to push away and move large organs, such as the stomach and the liver, by the tips of the arm portions 102 or the treatment tools 111. However, in the treatment method of this embodiment, in Step S2, since these organs move automatically due to gravity, even if the treatment endoscope 100 and the treatment tools 111 are used, a treatment can be performed without problems.

In addition, either Step S2 of performing a postural change or Step S1 of introducing the treatment tools into a body cavity may be first performed. That is, after anesthesia induction or the like, organs may be moved to a position where manipulation is easily performed by suitably setting the posture of the patient in advance, and then, the treatment tools may be inserted into the body cavity.

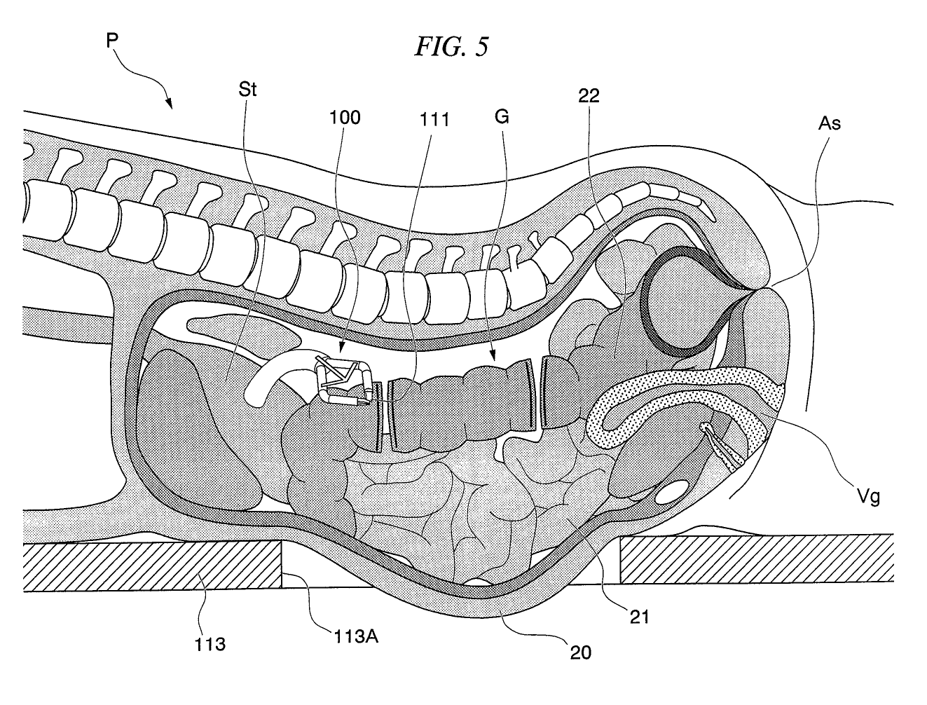

Subsequently, a second embodiment of the present invention will be described with reference to FIG. 5. The treatment method of this embodiment is different from the treatment method of the above first embodiment in that a portion of the abdominal wall is further moved downward when a postural change to a prone position is made.

In addition, in the following description, elements common to those of each embodiment which have already been described will be denoted by the same reference numerals, and duplicate description will be omitted.

FIG. 5 is a sectional view showing a state of the patient P in Step S2 of the treatment method of this embodiment. The treatment in this embodiment is the excision of intestines, and FIG. 5 shows as an example of the treatment a state where a descending colon, which is a portion of a large intestine, is excised.

A hole 113A is formed in a portion of a bed 113 on which the patient P lies, which corresponds to the abdominal wall. The hole 113A may be a bottomed hole, or may be provided as a through hole. When the posture of the patient P is changed to a prone position in Step S2, a portion of the abdominal wall 20 is suspended into the hole 113A, and moves to a position lower than other parts. Along with this, as shown in FIG. 5, the gap G is formed on the back side since the small intestine 21 and the large intestine 22 also move downward in the abdominal cavity.

In Step S3, the operator performs a desired manipulation on the large intestine 22, using the treatment tools 111 introduced via the treatment endoscope 100 from the stomach St, similarly to the first embodiment. At this time, since the gap G is formed, the descending colon can be easily excised. Note that the treatment tools 111 may be introduced from an anus As instead of the stomach St, or may be introduced from a vagina Vg if a patient is a woman, and an introduction passage may be suitably selected from these according to a part where a treatment is performed.

In the treatment method of this embodiment, the same effects as the above first embodiment can be obtained. Additionally, since a portion of the abdominal wall 20 moves to a position still lower than other parts, the gap G can be suitably formed even in a region where the volume of organs which occupy the body cavity is relatively large such as a region where the small intestine 21 and the large intestine 22 are received.

For this reason, it may be possible to skip a pneumoperitoneum step which is usually performed in the manipulation into the abdominal cavity without opening the abdominal cavity, depending on the contents of manipulation.

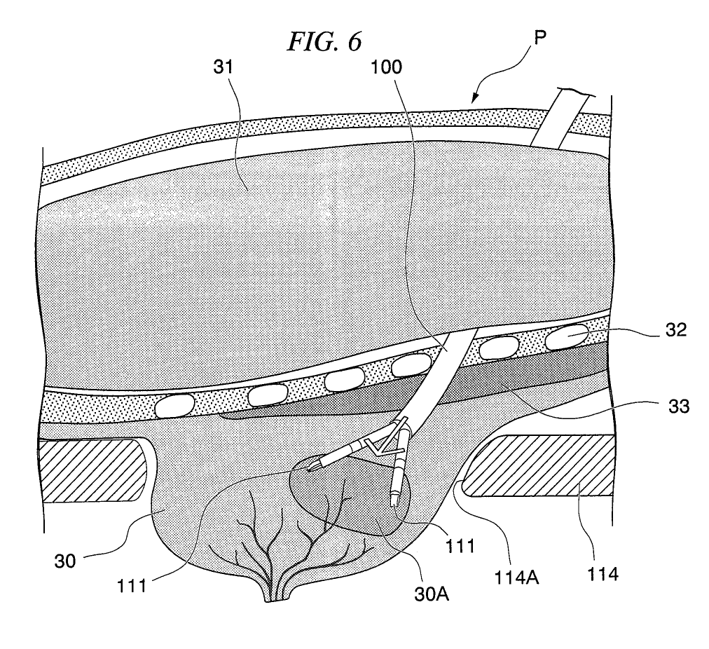

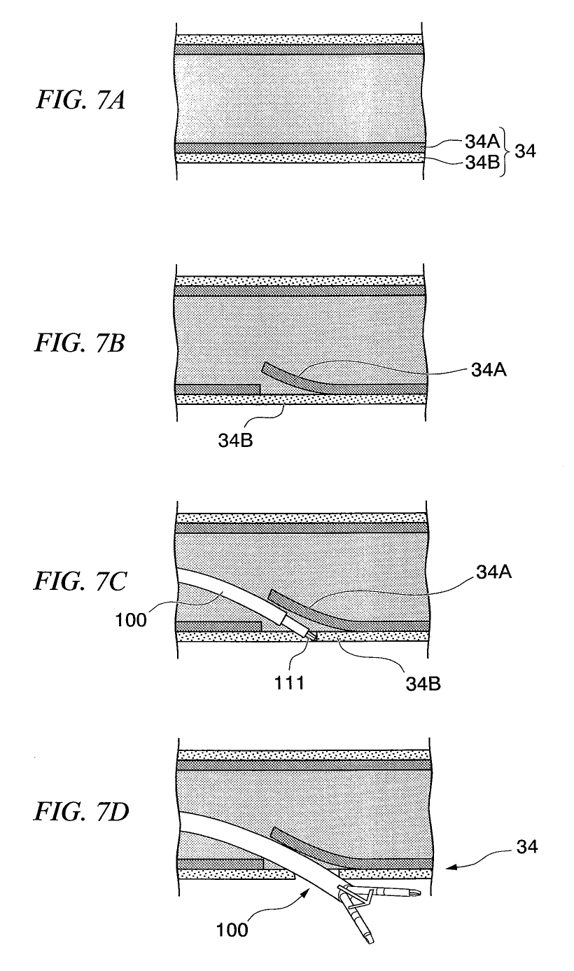

Subsequently, a third embodiment of the present invention will be described with reference to FIGS. 6 to 7D. The treatment method of this embodiment is different from the treatment methods of the above respective embodiments in that a portion of a mammary gland is further moved downward when a postural change to a prone position is made.

FIG. 6 is a sectional view showing a state of the patient P in Step S2 of the treatment method of this embodiment. The treatment in this embodiment is the excision of a breast cancer.

A hole 114A is provided in a portion of a bed 114 used in this embodiment, which corresponds to the mammary gland. Accordingly, when the posture of the patient P is changed to a prone position in Step S2, the mammary gland 30 advances into the hole 114A, and the mammary gland 30 moves to a part still lower than other parts.

In Step S1, the operator introduces the treatment tools 111 to the vicinity of a tumor 30A in the mammary gland 30, which is received within the hole 114A in a stable state, using the treatment endoscope 100. Since the mammary gland 30 is located closer to the head than the diaphragm, at the time of the approach of the treatment tools 111, as shown in FIG. 6, it is preferable first to reach a thoracic cavity via the esophagus or the like, and to reach the tumor 30A through a pectoralis major muscle 33 from the gap between ribs 32 or the like while avoiding a lung 31 or the like.

As the approach via the esophagus described above, for example, a well-known tunneling method can be appropriately employed. Hereinafter, the procedure of the tunneling method will be described.

First, as shown in FIG. 7A, a treatment is performed on the wall surface of an esophagus 34 having a mucous membrane layer 34A and a muscle layer 34B, and as shown in FIG. 7B, the mucous membrane layer 34A is peeled from the muscle layer 34B. The treatment at this time can be performed by injecting a saline solution or the like under the mucous membrane layer to separate the mucous membrane layer 34A from the muscle layer 34B and incising the mucous membrane layer by a high-frequency knife or the like, similarly to a mucous membrane peeling method performed in a stomach or the like. In addition, at this time, the mucous membrane layer 34A is not cut off, but is left in the shape of a valve.

Next, as shown in FIG. 7C, the treatment endoscope 100 is moved under the valve-like mucous membrane layer 34A, the muscle layer 34B is incised using the treatment tools 111 or the like, and as shown in FIG. 7D, the treatment tools advance into the thoracic cavity through the wall surface of the esophagus 34. After the manipulation, the hole of the wall surface of the esophagus 34 can be suitably closed by first closing a surgical incision of the muscle layer 34B with suturing, a clip, or the like, and closing a surgical incision of the mucous membrane layer 34A by suturing, a clip, or the like.

Although surgical manipulation on the mammary gland, such as excision of a breast cancer, is usually performed in a supine position, the shape of the mammary gland including a number of fat tissues is not easily stabilized in the supine position, and is not easy. According to the treatment method of this embodiment, since the mammary gland is fitted into the hole 114A by the postural change in Step S2, and its shape is held in a stable state, the position of the tumor 30A is also stabilized. Thus, the manipulation can be more reliably and safely performed.

Additionally, since the treatment tools 111 are introduced from the thoracic cavity side, the treatment tools can easily approach even a tumor in a deep part of the mammary gland 30 near the ribs 32. Accordingly, even in the tumor in the deep part, just the tumor can be excised without largely excising the mammary gland, and the deterioration in the QOL (quality of life) of a patient can be prevented.

In all the treatment methods of the first to third embodiments described above, the posture of the patient is set to a prone position in Step S2. However, such a treatment method is not limited to those of the first to third embodiments.

For example, also in the case of a kidney excision, an adrenal excision, a liver excision including a partial (especially a region on the back side) excision, or the like, since a gap is secured around a tissue to be treated by a postural change to a prone position, it is possible to suitably perform a treatment similarly to the first to third embodiments.

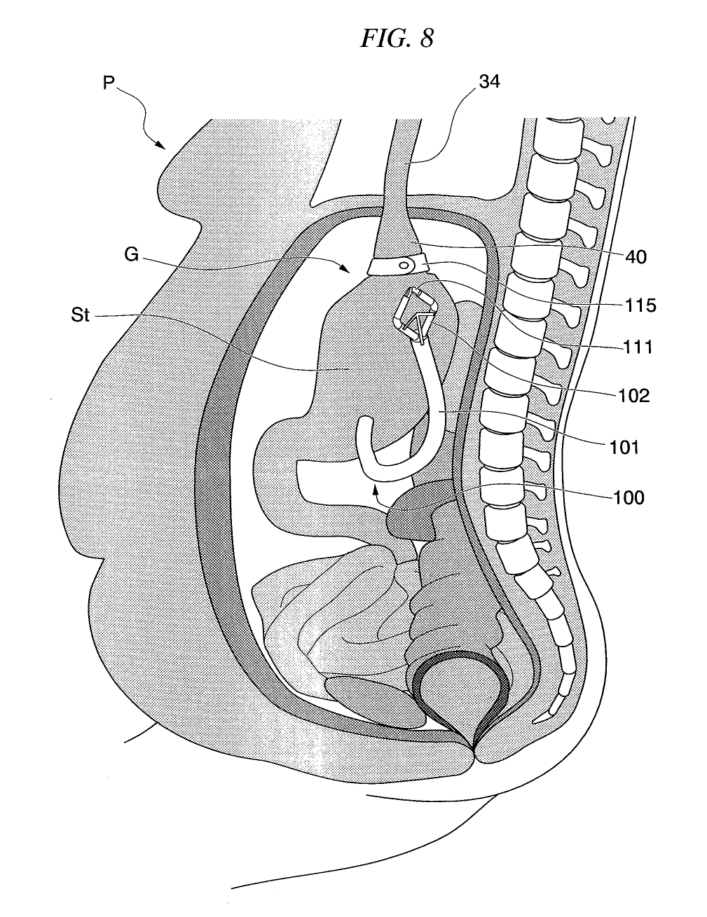

Subsequently, a fourth embodiment of the present invention will be described with reference to FIGS. 8 to 10D. The treatment method of this embodiment is different from the treatment methods of the above respective embodiments in that the posture of the patient is set to a seating position.

FIG. 8 is a sectional view showing a state of the patient P in Step S2 of the treatment method of this embodiment. The treatment in this embodiment is gastric reduction surgery for an obese patient, and FIG. 8 shows a view which conducts a stomach band operation as an example of the gastric reduction surgery.

Since the obese patient has a very thick intestine wall, the pneumoperitoneum step cannot be performed in many cases. In those cases, it is difficult to perform a normal operation under a laparoscope. Then, in the treatment method of this embodiment, in Step S2, as shown in FIG. 8, the posture of the patient P is changed and set to a seating position. The introduction of the treatment tools in Step S1 may be made before or after Step S2.

In Step S3, the operator makes the treatment endoscope 100 inserted from the mouth reach an abdominal cavity from the stomach St in a state where a stomach band 115 is gripped by the treatment tools 111 inserted through the arm portions 102, and introduces the treatment endoscope to the vicinity of a cardiac region 40 where the stomach band 115 is to be set.

Next, as shown in FIG. 8, the operator attaches the stomach band 115 to the periphery of the cardiac region 40 to narrowly tighten the inlet of the stomach St.

According to the treatment method of this embodiment, as the posture of the patient P is changed to a seating position, the stomach St is hung by the esophagus 34, and the gap G is secured around the stomach. Therefore, as compared with a case where manipulation is performed in a supine position without performing laparotomy, the stomach band 115 can be more easily set.

In the treatment method of this embodiment, the patient does not rotate, unlike a case where a postural change is made from a supine position to a prone position. Therefore, it is possible to approach the treatment tools, which are used in a normal operation under a laparoscope, not only from a natural opening such as the mouth, but also from a place where the abdominal wall is relatively thin, such as a navel. Since the stomach St is hung in this case, as compared with a case where manipulation is performed in a supine position, manipulation can be more easily performed.

Furthermore, as shown in FIG. 8, when the treatment endoscope 100 is introduced by way of the esophagus 34, the stomach band 115 is set in a state where the insertion portion 101 of the treatment endoscope 100 is inserted through the esophagus 34. Accordingly, since the insertion portion 101 of the treatment endoscope 100 serves as a core rod of the cardiac region 40, it is possible to prevent the cardiac region 40 from becoming too narrow by the stomach band 115, and the stomach band 115 can be more suitably set, which is preferable.

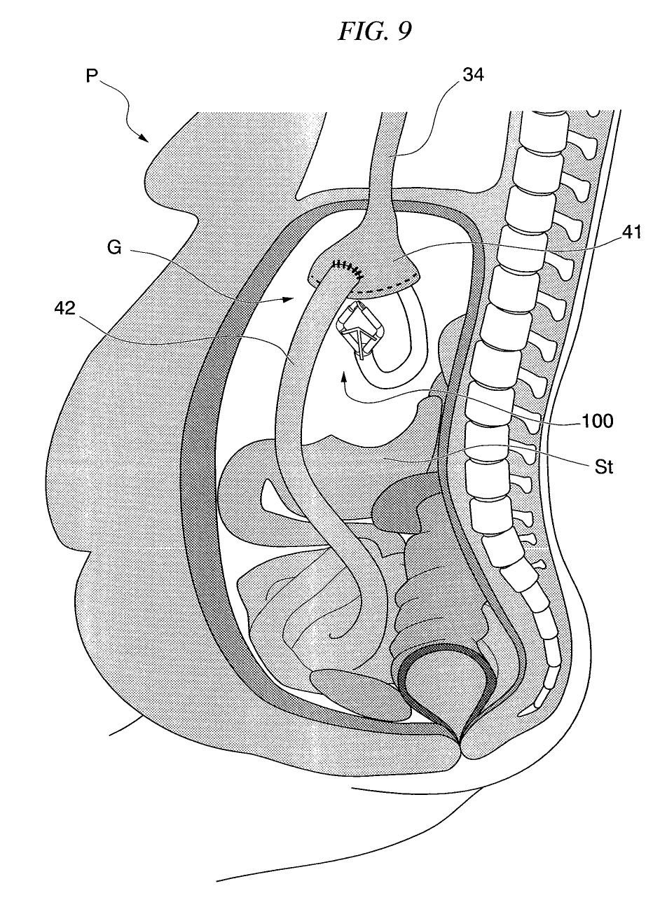

The treatment method of this embodiment for an obese patient is not limited to the above-described stomach band operation. For example, even in the Roux-en-Y method, when posture is changed to a seating position as shown in FIG. 9 after a great portion of the stomach St is cut off to form a porch (a remaining stomach) 41, the separated portion moves below, and Gap G is secured in the periphery of the porch 41. Accordingly, the anastomosis operation between the porch 41 and a jejunum 42, and the like, can be easily performed.

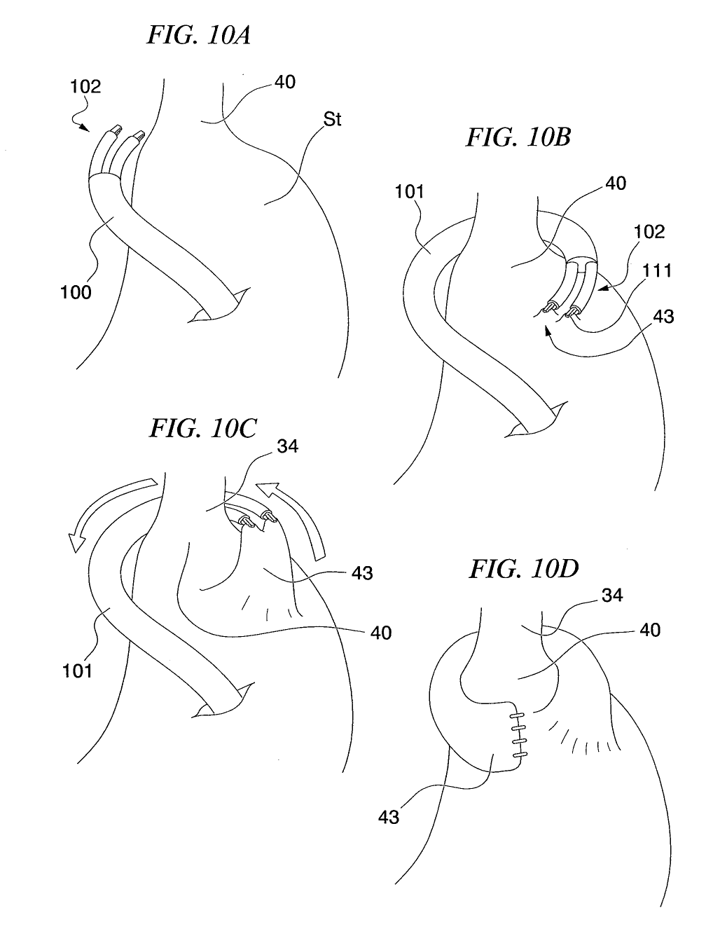

Moreover, the treatment method of this embodiment can also be applied to the Nissen method. FIGS. 10A to 10D are views showing the flow of manipulation when the treatment method of this embodiment is applied to the Nissen method. Hereinafter, the procedure will be described.

The operator first advances the treatment endoscope into an abdominal cavity from the stomach St similarly to the above-described stomach band operation, and as shown in FIG. 10A, makes the arm portions 102 of the treatment endoscope 100 arrive at the vicinity of the cardiac region 40.

Next, the operator operates the insertion portion 101 to make the insertion portion go around the cardiac region 40, and as shown in FIG. 10B, grips a stomach wall 43 by the treatment tools 111 at the tips of the arm portions 102.

Next, while the stomach wall 43 is gripped, as shown in FIG. 10C, the operator pulls back the insertion portion 101 so as to be retracted. The gripped stomach wall 43 is pulled by the treatment tools 111, and is wound around the cardiac region 40 and the esophagus 34 in the vicinity thereof.

Thereafter, the operator exchanges one of the treatment tools 111 as necessary, and as shown in FIG. 10D, fixes the stomach wall 43 wound around the cardiac region 40 by a suture thread, a clip, etc., to hold a state where the cardiac region 40 and the esophagus 34 are narrowed, thereby ending manipulation.

As described above, even in a case where the treatment method of this embodiment is applied to the Nissen method, since the posture of the patient is set to a seating position in Step S2, the stomach St is hung. Thus, manipulation is very easily performed. In addition, although a considerable amount of force is required for pulling of the stomach wall 43, the amount of force can be generated by pulling the insertion portion 101 and does not depend on the structure of the arm portions 102. Therefore, even if an apparatus having a flexible insertion portion is used like the treatment endoscope 100, the pulling can be performed without problems.

Additionally, although an example of the treatment method for an obese patient has been described in this embodiment, the treatment method of this embodiment can also be applied to a patient who is not obese without problems. Accordingly, in the case where the Roux-en-Y reconstruction method is performed on a stomach cancer patient, or in the case where the Nissen method is performed on a GERD (Gastroesophageal Reflux Disease) patient, the procedure can easily be performed by applying the treatment method of this embodiment.

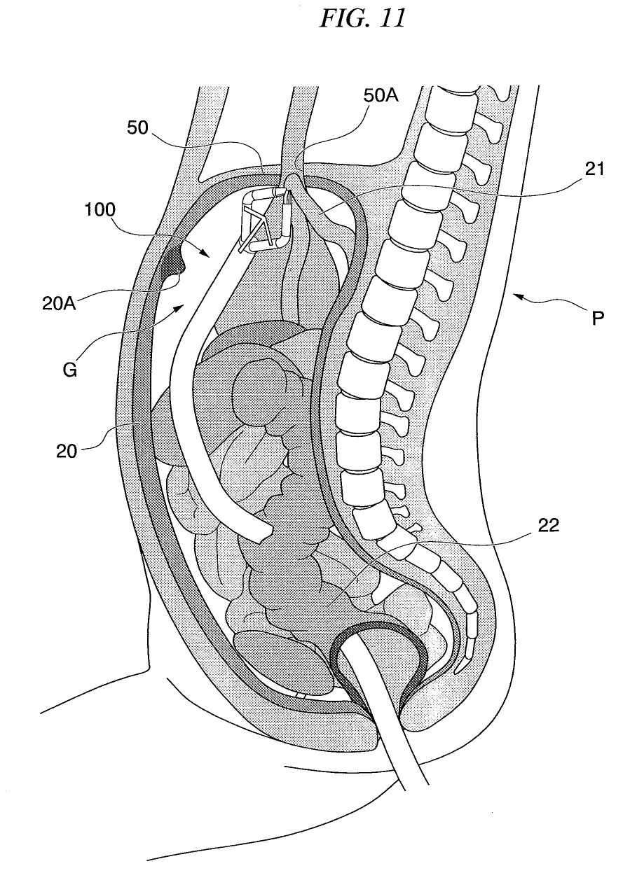

Subsequently, a fifth embodiment of the present invention will be described with reference to FIG. 11. The different points between the treatment method of this embodiment and the treatment methods of the above respective embodiments are an introduction passage of a treatment tool, and the position of a tissue to be treated.

FIG. 11 is a sectional view showing a state of the patient P in Step S2 of the treatment method of this embodiment. In this embodiment, the introduction of the treatment tools 111 into an abdominal cavity in Step S1 is performed by way of the large intestine 22.

In Step S2, similarly to the above-described fourth embodiment, the posture of the patient P is changed to a seating position, and the gap G is secured in the region of the abdominal cavity in the vicinity of the diaphragm 50 by the downward movement of organs.

In such a state, manipulation can be suitably performed on a tumor 20A formed inside the abdominal wall 20, the hernia in which the small intestine 21 has advanced into an esophageal opening 50A of the diaphragm, or the like. Particularly when a tissue to be treated exists in a position like the tumor 20A, and a treatment method of making a hole in the abdominal wall like a normal operation under a laparoscope is adapted, the approach to the target tissue is difficult unless the treatment tools are reversed about 180 degrees from the advancement direction. Therefore, substantial time is required until treatment is started. In the treatment method of this embodiment, the treatment tools can be introduced to the vicinity of the tumor 20A from below at a reasonable angle. Therefore, manipulation can be very easily performed.

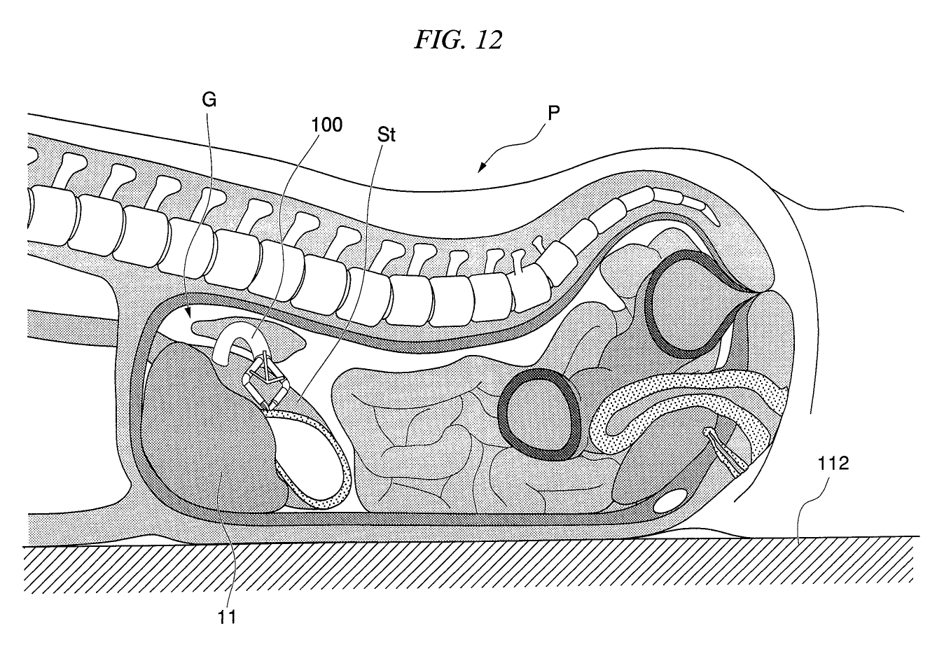

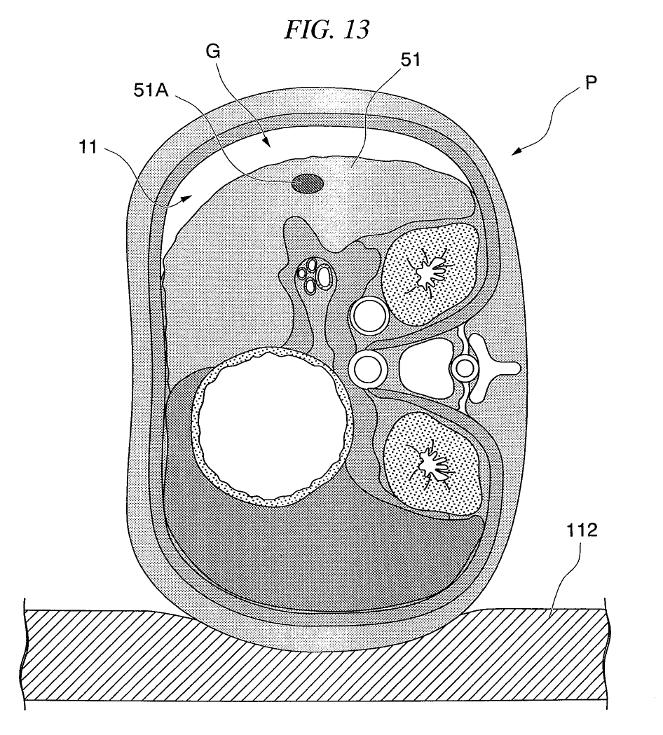

Subsequently, a sixth embodiment of the present invention will be described with reference to FIGS. 12 to 13. The treatment method of this embodiment is different from the treatment methods of the above respective embodiments in that the movement of organs by posture setting is performed twice.

FIG. 12 is a sectional view showing a state of the patient P in Step S2 of the treatment method of this embodiment. The manipulation in this embodiment is partial excision of the liver.

First, the operator introduces the treatment tools 111 into an abdominal cavity via the stomach St, using the treatment endoscope 100 in Step S1. Then, in Step S2, the operator first performs a first postural change to make the patient P take a prone position, as shown in FIG. 12. In the prone position, since the gap G is secured on the back side as described above, manipulation is performed using the gap G.

Here, when a tissue to be treated, such as a tumor, is located in a position close to a flank, such as the left end of a left lobe or the right end of a right lobe in the liver 11, there is a possibility that manipulation cannot be performed sufficiently only by the gap G secured on the back side.

In such a case, the operator performs a second postural change to make the patient take a lateral recumbent position, as shown in FIG. 13. Whether either the right or the left is turned down may be suitably determined according to the position of a tissue to be treated, for example, when a tumor is located in the right lobe, the left is turned down. Hereinafter, a state where the left is turned down will be described as an example. When the posture of the patient is changed to a lateral recumbent position, organs move to the left (downward in FIG. 13) of the body of the patient by gravity, and the gap G is secured on the right flank side which becomes the upside in the lateral recumbent position. In this way, the operator can appropriately perform treatment on a tumor 51A of a right lobe 51 that is a tissue to be treated.

According to the treatment method of this embodiment, two postural changes are performed according to the progress of a treatment so that a gap is secured in a desired position. Thus, manipulation can be more suitably performed.

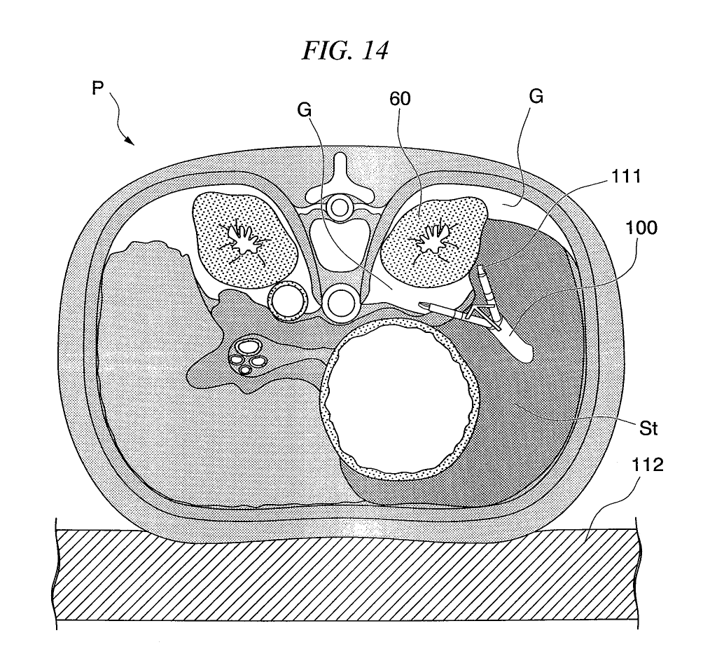

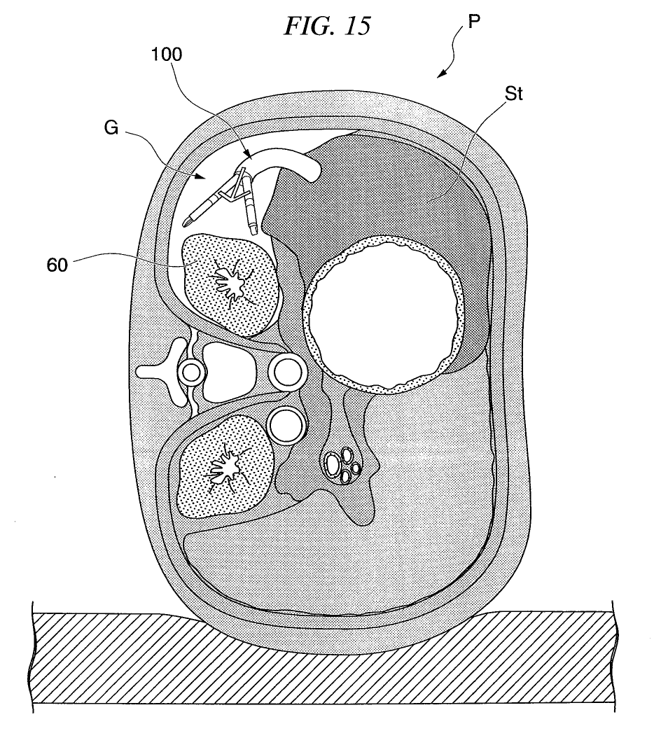

Subsequently, a seventh embodiment of the present invention will be described with reference to FIGS. 14 and 15. The different point between the treatment method of this embodiment and the treatment methods of the above respective embodiments is a tissue to be treated.

FIG. 14 is a sectional view when seeing a state of the patient P in Step S2 of the treatment method of this embodiment from the head side. The manipulation in this embodiment is the extirpation of the left kidney.

The operator introduces the treatment tools 111 into a left kidney 60 within an abdominal cavity, using the treatment endoscope 100 in Step S1. As the introduction passage, introduction via the stomach is the simplest.

Since the kidney including the left kidney 60 is fixed to the back side, a sufficient gap is not provided around the kidney, which makes treatment difficult. Thus, the operator makes the patient P take a prone position as a first postural change, as shown in FIG. 14, in Step S2. Since the gap G is secured on the back side and right side of the left kidney 60 (the upside and left side of the left kidney 60 in FIG. 14) by the movement of organs, the operator performs various treatments, such as peeling and blood vessel processing, on the left kidney 60 using the gap G.

In the prone position, a gap is not sufficiently formed on the left side of the left kidney 60 due to the positional relationship with other organs. After the operator has ended the above-described various treatments, as shown in FIG. 15, the operator makes the patient P take a lateral recumbent position where the left flank is turned up as the second postural change. Thereby, the gap G is secured on the left side of the left kidney 60, and the operator performs treatments on a region where peeling, blood vessel processing, or the like has not been performed yet, using Gap G. In this way, the treatment can be suitably performed.

Although the preferable embodiments of the present invention have been described hitherto, the present invention is not limited to the embodiments. Additions, omissions, substitutions, and other modifications can be made without departing from the spirit or scope of the present invention.

For example, in the above-described embodiments, the prone position, the seating position, and the lateral recumbent position have been described as the examples of postural changes. However, the postural changes in the treatment method of the present invention are not limited thereto.

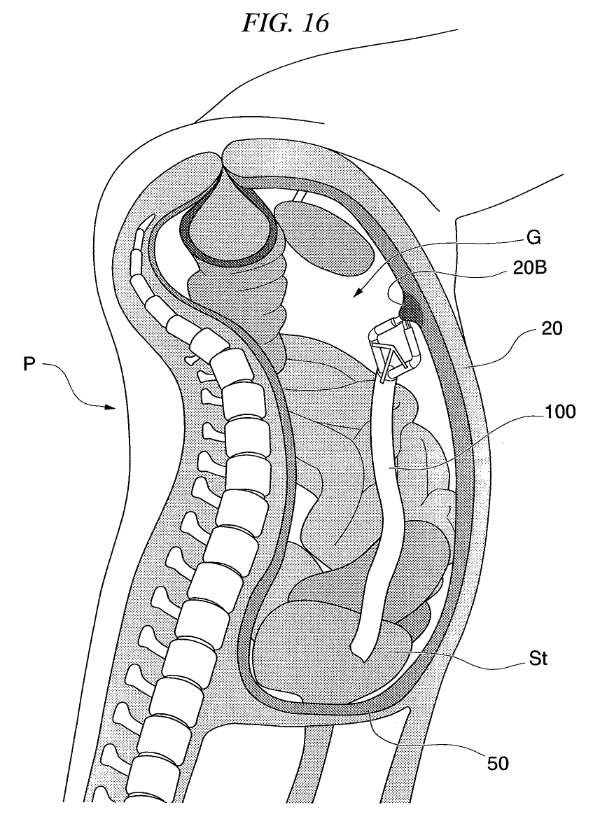

For example, as shown in FIG. 16, when a tumor 20B is located inside the abdominal wall 20 in the vicinity of the lower abdomen, the operator may change the posture of the patient P to an inverted position in which the head is turned down, and perform manipulation. In this case, since organs, such as a small intestine and a large intestine, move to the vicinity of the diaphragm 50 and the gap G is formed around the tumor 20B, manipulation can be appropriately performed.

Additionally, the postural change may be performed three times or more. Here, since the introduction of the treatment tools from holes other than a natural opening becomes difficult as the kinds of the postural change increase, it is preferable to introduce the treatment tools via the natural opening using the treatment endoscope 100 or the like. When the treatment endoscope 100 is used, the arm operating portion 105 and the endoscope operating portion can be arranged in a position apart from a patient. Thus, there are also merits that various kinds of piping, such as a tube for intravenous drip and a tracheal tube, which are attached to the patient hardly become an obstacle to the operator compared with an operation under a laparoscope or the like.

What is claimed is:

1. A treatment method for a tissue in a body cavity of a patient, the treatment method comprising the steps of:
endoscopically introducing a treatment tool into the body cavity of the patient from a natural opening of the patient; and
setting the posture of the patient to a prone position.

2. A treatment method for a tissue in a body cavity of a patient, the treatment method comprising the steps of:
changing the posture of the patient to move organs in a thoracic cavity and an abdominal cavity of the patient so as to form a space;
endoscopically introducing a treatment tool into the body cavity of the patient from a natural opening of the patient or a pierced hole formed in a body wall of the patient; and
making the treatment tool approach organs in at least one of the thoracic cavity and the abdominal cavity using the space.

3. The treatment method according to claim 2,
wherein in the changing step, the patient is made to take a seating position.

4. The treatment method according to claim 2,
wherein in the changing step, the body side of the patient which becomes lower side in a recumbent position is changed.

5. The treatment method according to claim 2,
wherein in the changing step, the patient is made to take an inverted position.

6. An operation method, comprising the step of introducing a plurality of endoscopes
into a body cavity of a patient.

7. The operation method according to claim 6, further comprising a treatment step of inserting a treatment tool into at least one of the plurality of endoscopes, and treating a target part in the body cavity of the patient.

8. The operation method according to claim 6,
wherein at least one of the plurality of endoscopes is introduced into the body cavity of the patient from a natural opening of the patient.

9. The operation method according to claim 6,
wherein all the plurality of endoscopes are introduced into the body cavity of the patient from a natural opening of the patient.

10. The operation method according to claim 7,
wherein the plurality of endoscopes are introduced into the body cavity of the patient from a plurality of different natural openings of the patient, respectively,
in the treatment step, a target part in an abdominal cavity of the patient is treated, and
another endoscope of the plurality of endoscopes is introduced into a thoracic cavity of the patient.

11. The operation method according to claim 6,
wherein the plurality of endoscopes are introduced into the body cavity of the patient from a plurality of different natural openings of the patient, respectively, and are made to approach the same cavity in the body cavity.

12. The operation method according to claim 7,
wherein the plurality of endoscopes are introduced into the body cavity of the patient from a plurality of different natural openings of the patient, respectively, and
in the treatment step, separate target parts of the patient are treated by the plurality of endoscopes.

13. The operation method according to claim 7,
wherein the plurality of endoscopes are introduced into the body cavity of the patient from a plurality of different natural openings of the patient, respectively, and
in the treatment step, the same target part of the patient is treated by the plurality of endoscopes.
